(19) United States
(12) Patent Application Publication
(10) Pub. No.: US 2022/0323700 A1
(43) Pub. Date: Oct. 13, 2022

(54) TRACKING RESPIRATORY MECHANICS OF A PATIENT

(71) Applicant: KONINKLIJKE PHILIPS N.V., EINDHOVEN (NL)

(72) Inventors: Albertus Cornelis den Brinker, Eindhoven (NL); Cornelus Hendricus Bertus Arnoldus van Dinther, Eindhoven (NL); William Anthony Truschel, Oakmont, PA (US); Tamara Mathea Elisabeth Nijsen, Eindhoven (NL); Samer Bou Jawde, Cambridge, MA (US)

(21) Appl. No.: 17/671,898

(22) Filed: Feb. 15, 2022

Related U.S. Application Data
(60) Provisional application No. 63/173,858, filed on Apr. 12, 2021.

(51) Int. Cl. A61M 16/00; G16H 20/40; G16H 40/63; G16H 50/20

(52) U.S. Cl. CPC A61M 16/026; A61M 16/0051; G16H 20/40; G16H 40/63; G16H 50/20; A61M 2230/42

(57) ABSTRACT

An apparatus, comprising a processor and memory storing instructions that, when executed by the processor, cause the processor to: receive ventilator data obtained from continual mechanical ventilatory support of a patient over a period of time, analyze the ventilator data to identify a plurality of breathing cycles, classify the plurality of breathing cycles into normal breathing cycles and abnormal breathing cycles, using a machine learning algorithm, detect a change in the normal breathing cycles, compared to normal breathing cycles identified by the apparatus based on ventilator data obtained from continual mechanical ventilatory support of the patient over a previous period of time, and generate output indicative of the change in the normal breathing cycles.

### CROSS-REFERENCE TO RELATED APPLICATIONS

This patent application claims the priority benefit under 35 U.S.C. § 119(e) of U.S. Provisional Application No. 63/173,858, filed on Apr. 12, 2021, the contents of which are herein incorporated by reference.

### FIELD OF THE INVENTION

The disclosure herein relates to tracking the respiratory mechanics of a patient.

### BACKGROUND OF THE INVENTION

Besides their primary use of aiding patients in breathing, mechanical ventilators typically control and measure ventilation parameters such as tidal volume and peak inspiratory pressure as well as record ventilation waveforms such as pressure and flow, on the basis of which respiratory mechanics such as resistance and compliance may be derived. Analysis of such respiratory mechanics may allow a clinician to monitor pulmonary diseases' status and progression in patients closely. In addition, such analysis may be used to aid clinicians in diagnosing diseases, making clinical decisions, measuring the effects of treatments such as medicine intake, and adjusting mechanical ventilator settings to specific patient needs. However, current techniques of analyzing respiratory mechanics are limited since they are typically assessed based on measurements obtained intermittently or under specific conditions. For example, ventilation parameters may be obtained by requiring clinicians to perform hold maneuvers during invasive ventilation in an intensive care unit, or require minimal patient effort, i.e., sedation of the patient. However, as a single intermittent measurement, such hold maneuvers may not be representative of a patient's respiratory mechanics. Even if they are representative at the moment of measurement, it may not be easy or possible to accurately detect the occurrence of changes in the respiratory mechanics over time using such measurements. Therefore, it would be desirable to detect changes in the respiratory mechanics from measurements obtained during continual ventilatory support of the patient and avoid as much as possible the additional burden to the monitored patient. Furthermore, based on continuous tracking, it is beneficial to define a certain pattern across a specific time period and assess how and when this pattern alternates and in which direction (deterioration or improvement).

### SUMMARY OF THE INVENTION

According to an aspect, there is provided an apparatus, comprising a processor and memory storing instructions that, when executed by the processor, causes the processor to: receive ventilator data obtained from continual mechanical ventilatory support of a patient over a period of time, analyze the ventilator data to identify a plurality of breathing cycles, classify the plurality of breathing cycles into normal breathing cycles and abnormal breathing cycles, using a machine learning algorithm, detect a change in the normal breathing cycles, compared to normal breathing cycles identified by the apparatus based on ventilator data obtained from continual mechanical ventilatory support of the patient over a previous period of time, and generate an output indicative of the change in the normal breathing cycles.

In this way, gradual changes over time to the normal breathing cycles of the patient, which are indicative of gradual changes over time to the respiratory mechanics of the patient, may be detected.

In one embodiment, the instructions further cause the processor to: generate a respiratory model specific to the patient, based at least on the received ventilator data corresponding to the normal breathing cycles. Models may be easier to work with for particular tasks, such as visualizing the changes in breathing cycles (respiratory mechanics) that occur gradually over time. The respiratory model may also use ventilator data obtained from continual mechanical ventilatory support of the patient (i.e., tidal volume) over the previous period of time. Thus, a patient-specific progression of the breathing cycles (respiratory mechanics) may be modeled. In some cases, ventilator data corresponding to the abnormal breathing cycles may be used additionally to generate the model. The respiratory model may be a waveform model.

While analysis of the normal breathing cycles may reveal gradual changes over time to the respiratory mechanics of the patient, analysis of the abnormal breathing cycles may also provide insights into the respiratory mechanics of the patient or any other situation (e.g., patient-ventilator asynchrony, coughing, and so so). Thus, in some embodiments the instructions further cause the processor to: analyze the abnormal breathing cycles, using at least one of: the normal breathing cycles of the patient over the period of time as a reference, predetermined data describing breathing cycles, and a predetermined respiratory model describing patient breathing cycles. Using the normal breathing cycles as a reference may be advantageous because deviations from the normal breathing cycles, such as coughing or secretions, may be more accurately determined in relation to the normal breathing cycles. The analysis may include classifying the abnormal breathing cycles into different types of abnormal breathing cycles. The apparatus may generate output indicative of the analysis. For example, the output may indicate a deviation of an abnormal breathing cycle from the normal breathing cycles, or may indicate a type of abnormal breathing cycle.

Any suitable machine learning algorithm may be used. Since ventilator data may be obtained from continual mechanical ventilatory support over hours or even days, in one embodiment only some of the ventilator data is labeled, while in another embodiment, the ventilator data is completely unlabeled. Thus, the machine learning algorithm may be a semi-supervised or unsupervised machine learning algorithm. One example of machine learning is a clustering algorithm. Thus, in one embodiment, to classify the plurality of breathing cycles into normal and abnormal breathing cycles, the instructions cause the processor to: extract values of a parameter from the received ventilator data, the parameter comprising at least one of a lung parameter and a ventilator parameter, apply the machine learning algorithm to the extracted values, to obtain a plurality of groups of extracted values, and designate at least one group of extracted values as representing the normal breathing cycles, and at least one group of extracted values as representing the abnormal breathing cycles.

In one embodiment, the groups (subsets) of extracted values are designated as normal and abnormal breathing cycles based on at least one of: a numeric property of each of the groups, a manually generated annotation of the ventilator data, and predetermined data describing a relationship between values of the parameter and types of breathing cycle. For example, the numeric property may comprise the size of the group, based on the assumption that the normal breathing cycles may be the predominant breathing cycle.

The received ventilator data may be subjected to additional processing in order to identify the breathing cycles. In one embodiment, to identify the plurality of breathing cycles from the received ventilator data the instructions cause the processor to: segment the received ventilator data into a plurality of data segments, each data segment corresponding to an individual breathing cycle. Thus, the apparatus may be used with various mechanical ventilators that provide ventilator data in different forms.

In one embodiment, the instructions further cause the processor to: identify a fraction of the breathing cycles in the ventilator data that are normal breathing cycles, the output data including an indication of the fraction. Counting the number of normal and abnormal breathing cycles may be easily done and may provide a valuable, even if somewhat coarse, way of detecting or predicting respiratory events.

In one embodiment, the ventilator data is received in real time or near real time. In this way, at least some of the other method steps including, for example, the generation of the output data and/or the generation of the breathing model, may be carried out in real time. Of course, there may be a short delay between receiving the ventilator data and processing the ventilator data to allow a statistically significant number of normal breathing cycles to be obtained. Furthermore, measuring/calculating parameters in real time may be useful to monitor sudden events such as coughing, but also to monitor slow changing of resistance or compliance of the respiratory system of the patient.

The output may take various forms. For example, in one embodiment, the instructions may further cause the processor to: determine a change in a respiratory status of the patient based on the change in the normal breathing cycles, and the output may be indicative of the change in the respiratory status. Such an indication may simply inform that the respiratory status of the patient is deteriorating or improving, or it may provide more detailed information such as the progression of a disease. The output may comprise a visual representation of the change in the normal breathing cycles, such as superimposed waveforms of the normal breathing cycles for the current and at least one previous period of time. In cases where a breathing model is generated, the breathing model may be the output. In another embodiment, the output comprises an alarm signal (e.g., visual and/or audible) indicating that the change in the normal breathing cycles satisfies an alarm condition. The apparatus may be provided as a stand-alone apparatus or integrated with another apparatus. For example, the apparatus may be connected to (indirectly, such as via a network, or directly) a mechanical ventilator or integrated with a mechanical ventilator.

According to another aspect, there is provided a computer-implemented method, comprising: receiving ventilator data obtained from continual mechanical ventilatory support of a patient over a period of time; analyzing the ventilator data to identify a plurality of breathing cycles; classifying the plurality of breathing cycles into normal breathing cycles and abnormal breathing cycles, using a machine learning algorithm; detecting a change in the normal breathing cycles, compared to normal breathing cycles identified based on ventilator data obtained from continual mechanical ventilatory support of the patient over a previous period of time; and generating an output indicative of the change in the normal breathing cycles.

Any of the methods, functions and processes described herein may be embodied as machine readable instructions stored on a computer readable medium, which may be non-transitory such as hardware storage devices (e.g., RAM (random access memory), ROM (read only memory), EPROM (erasable, programmable ROM), EEPROM (electrically erasable, programmable ROM), hard drives, and flash memory). Thus, according to another aspect, there is provided a computer readable medium storing instructions that, when executed by a processor, cause the processor to: receive ventilator data obtained from continual mechanical ventilatory support of a patient over a period of time, analyze the ventilator data to identify a plurality of breathing cycles, classify the plurality of breathing cycles into normal breathing cycles and abnormal breathing cycles using a machine learning algorithm, detect a change in the normal breathing cycles, compared to normal breathing cycles identified based on ventilator data obtained from continual mechanical ventilatory support of the patient over a previous period of time, and generate output indicative of the change in the normal breathing cycles.

These and other aspects will be apparent from and elucidated with reference to the embodiment(s) described hereinafter.

### BRIEF DESCRIPTION OF THE DRAWINGS

Exemplary embodiments will now be described, by way of example only, with reference to the following drawings, in which:

FIG. 1 is a functional block diagram of an apparatus for determining changes in breathing cycles;

FIG. 2 is flow diagram of a method of determining changes in breathing cycles;

FIG. 3 is flow diagram of a method of designating normal and abnormal breathing cycles using a machine learning algorithm;

FIG. 4 is a block diagram of a hardware implementation of the apparatus of FIG. 1;

FIG. 5 is an illustration of simulated ventilator data;

FIG. 6 is an illustration of breath setting and threshold analysis performed on the simulated ventilation data of FIG. 5;

FIG. 7 is an illustration of respiratory mechanics obtained from the simulated ventilator data of FIG. 5;

FIG. 8 is an illustration of time-binned data obtained from the analysis of FIG. 6; and

FIG. 9 is an illustration of time-binned data obtained from the data of FIG. 8.

### DETAILED DESCRIPTION

In general terms, there is provided an apparatus and a method for tracking respiratory mechanics of a patient over time as reflected by changes in breathing cycles from one period of time to another, where the breathing cycles are identified in ventilator data obtained from continual mechanical ventilatory support of the patient.

The term “breathing cycle” is used herein to refer to the combination of one inspiratory phase and one expiratory phase by the patient. Breathing cycles may be classified as “normal breathing cycles” and “abnormal breathing cycles”. In this context, the term “normal” is a relative term, i.e., depending on the respiratory state of the patient at a given time, so that a breathing cycle may be defined as “normal” with respect to breathing cycles that are statistically the same or statistically different from that breathing cycle.

The term “continual” or “continually” is not used to suggest that the mechanical ventilatory support of the patient is never interrupted or that hold maneuvers are not performed at all by the patient during the period of time. Rather, the term is used to indicate that mechanical ventilatory support is provided to the patient most of the time. Thus, in some cases, interruptions or hold maneuvers (or both) may occur during the period of time over which the mechanical ventilator support is provided to the patient. However, in other cases, uninterrupted mechanical ventilatory support may be provided and possibly with no-hold maneuvers. This may depend on the type of mechanical ventilator, some of which can only calculate parameters such as resistance and compliance when the patient performs a hold maneuver.

The term “period of time” may be defined in terms of a length of time, for example at least one hour, at least 12 hours, at least 24 hours, at least 48 hours, or longer. Alternatively, the period of time may be defined in terms of breathing cycles, for example, at least 10 breathing cycles, at least 20 breathing cycles, at least 50 breathing cycles, at least 100 breathing cycles, or more. Longer periods of time may provide more statistically significant data. Periods of time may have the same duration or may have different durations. Furthermore, periods of time may partially overlap.

The term “change” when referring to breathing cycles means that a measured or calculated parameter value of a breathing cycle is statistically different from a corresponding measured or calculated parameter value of another breathing cycle.

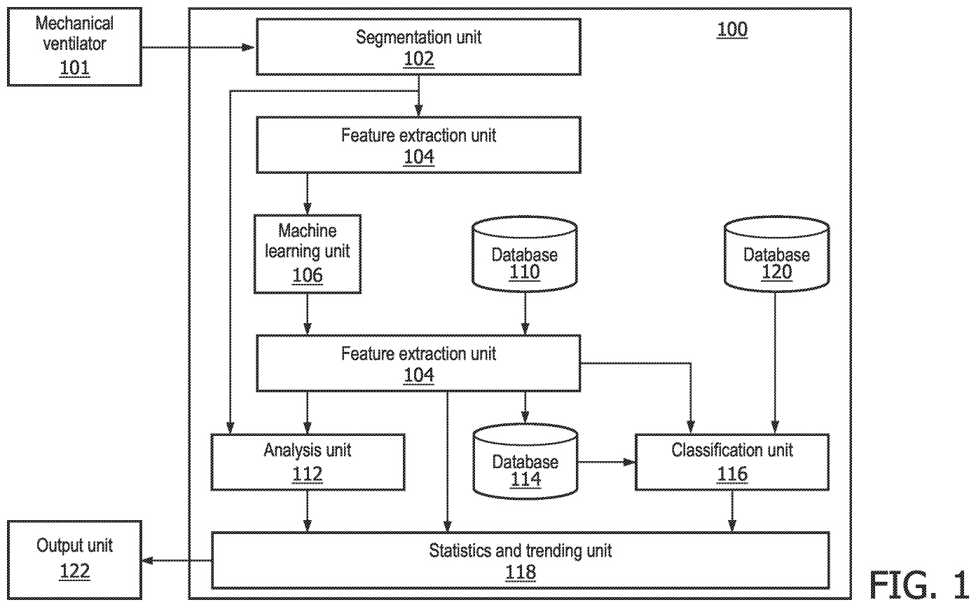

FIG. 1 illustrate an apparatus 100 for tracking respiratory mechanics of patient. The apparatus 100 obtains ventilator data from the mechanical ventilator 101. The ventilator data may be obtained from the mechanical ventilator 101 in real time or near real time, but this need to be the case. The ventilator data may comprise pressure and flow values. From these measured values, the volume may be calculated. Volume values may be calculated by the mechanical ventilator 101 or by the apparatus 100.

If the ventilator data obtained from the mechanical ventilator 101 has not already been segmented into breathing cycles by the mechanical ventilator 101, then this may be carried out by segmentation unit 102. For example, the segmentation unit 102 may analyze the waveform of at least one of pressure, flow, and volume to identify the breathing cycles.

Feature extraction unit 104 may extract values of a variety of parameters from the obtained ventilator data. Such parameters may include lung parameters and ventilation parameters. Examples of ventilation parameters include respiratory rate (RR), tidal volume (VT), and positive end-expiratory pressure (PEEP). Examples of lung parameters include respiratory compliance and resistance. For example, compliance and resistance may be determined from the waveform of at least one of pressure, flow, and volume. However, it will be appreciated that, in some examples, values of at least some of the parameters may be obtained directly from the mechanical ventilator 101.

Extracted values of the parameter(s) are input to the machine learning algorithm unit 106, which classifies the breathing cycles identified by the segmentation unit 102 into normal breathing cycles and abnormal breathing cycles using a machine learning algorithm. The classification may become more accurate if the machine learning algorithm is applied to values of two or more parameters, e.g., values of two or more lung parameters, values of two or more ventilation parameters, or a combination of values of at least one lung parameter and values of at least one ventilation parameter. A combination of lung and ventilation parameters may be particularly advantageous. This is because the lung parameters depend on the ventilation parameters. Thus, the combination of lung and ventilation parameters may allow this dependence to be observed and may allow the apparatus to better define what is the normal breathing cycle. Where values of two or more parameters are input to the machine learning algorithm unit 106, the machine learning algorithm may be applied once to the values of the two or more parameters. This is irrespective of whether the two or more parameters comprise lung parameters, ventilation parameters, or a combination of lung and ventilation parameters. The output of the machine learning algorithm may therefore provide one result on breath classification. However, the possibility that the machine algorithm may be applied separately for each parameter, while more complex, is not excluded. Any suitable machine learning algorithm may be used. In some examples the machine learning algorithm is an unsupervised learning algorithm, for example a clustering algorithm such as a K-means clustering. Broadly speaking, clustering algorithms group data points into clusters, or groups, so that data points within each group are similar to each other in some way. The similarity may be determined based on distance or density between data points. Thus, the machine learning algorithm unit 106 may classify the extracted values of the parameter(s) into clusters.

As noted above, the values of two or more parameters may be combined into one data set so that the machine learning algorithm unit 106 may output clusters that have values of both ventilation and lung parameters. At least one cluster may be designated as the normal breathing cycle (normal patient-specific behavior) by the selection unit 108. By way of simplified explanation, and not limitation, one cluster formed from two types of parameters with specific values/range may be designated as the normal breathing cycle, and one cluster with the same parameters but with different values/range may be designated as the abnormal breathing cycle. The normal breathing cycle may be defined with respect to other breathing cycles in a set of breathing cycles. For example, the normal breathing cycle may be the breathing cycle having similar features (values of a parameter at specific ventilator data) to most of the other breathing cycles in the set, i.e., based on frequency of occurrence. In the context of a clustering algorithm, the selection unit 108 may determine that a breathing cycle or breathing cycles with features belonging to the largest cluster may represent the normal breathing cycle. Other clusters may be defined as abnormal breathing cycles. The normal breathing cycle may also, or alternatively, be identified based on obtained mechanical ventilator settings and/or prior knowledge of expected respiratory behavior (i.e., predetermined data designated as normal breathing cycles) stored in a database 110. For example, a breathing cycle may be identified as the normal breathing cycle if the features of the breathing cycle match breathing cycle features that are stored in the database 100 and that have been designated as corresponding to a normal breathing cycle. In such case, the cluster with features corresponding the identified breathing cycle may be defined as corresponding to the normal breathing cycles.

The apparatus 100 may store the data corresponding to the breathing cycles in a database 114. Such data may be stored in sampled form, or in compacted form such as a prototype or a model that is created by the apparatus 100. The model may be a mathematical description of a waveform of the normal breathing cycle. The model may be considered to be patient-specific since it may be created based on parameters obtained from mechanical ventilator data of one patient.

The abnormal breathing cycles may be further analyzed and classified by classification unit 116. The classification unit 116 may use data describing characteristic events such as mucus secretion, coughing, asynchrony, etc. Such data may comprise predetermined data stored in a database 120. The data may also comprise the patient-specific data corresponding to the breathing cycles determined by the selection unit 108 and stored in the database 114. Thus, identified normal breathing cycles of the patient may be used as a reference.

An analysis unit 112 may extract values of a parameter from the ventilator data, in as far these they are not explicitly captured in the features obtained by the feature extraction unit 104.

Data from the selection unit 108, the analysis unit 112 the classification unit 116, and, may be input to the statistics and trending unit 118. The statistics and trending unit 118 may detect changes in the normal breathing cycles over time. For example, the statistics and trending unit 118 may determine breathing cycle statistics per hour, per a given number of breathing cycles, or per session. The statistics and trending unit 118 may also determine trends in the data over days, weeks, or even longer time periods, as well as clinical interventions (i.e. mucus clearance via sucking). For example, the apparatus 100 may identify a normal breathing cycle in a period of time occurring before a clinical intervention, identify a normal breathing cycle in a period of time occurring after the clinical intervention, and determine whether there is change in the normal breathing cycle. This may give an indication of the effectiveness of the clinical intervention.

Data from the statistics and trending unit 118 may be transmitted to an output unit 122. The output may simply indicate that the normal breathing cycle has changed. For example, the output may comprise data corresponding to the normal breathing cycles. Such data may be superimposed or otherwise concurrently displayed to allow the change to be visually observed. Alternatively the output may provide an indication of what the change represents in terms of a respiratory status of the patient. For example, the respiratory status may indicate, for example, the number of secretions, the number of coughs per night over an extended period of time. It may also indicate that the respiratory status of the patient has deteriorated or improved. This may involve determining an increase or decrease in the magnitude of a measured or calculated parameter value of the normal breathing cycles as a function of time. Such changes in measured or calculated parameter values may be correlated with progression of a disease, for example based on prior knowledge. The output may therefore also provide an indication of progression of a disease. Other examples of output include data describing a percentage of the different classes of breathing cycles in absolute or relative numbers, including their evolution over time. The output may comprise visual and/or audible alarm signals. These may be associated with changes in measured or calculated parameter values, for example if the increase or decrease in the magnitude goes above or below a threshold.

Whilst the apparatus 100 and the mechanical ventilator 101 are shown as separate entities, it will be realised that they may be configured as a single entity. Furthermore, while the database 110, 114, 120 are shown as being part of the apparatus 100, any of these databases may be separate entities that the apparatus 100 may access for example via a network.

The apparatus of FIG. 1 may be implemented in hardware, software, or a combination of hardware and software. Accordingly, each “unit” may be a software element or a hardware element such as a field-programmable gate array (FPGA) or an application-specific integrated circuit (ASIC), and performs a certain function. However, the term “unit” is not limited to software or hardware. The “unit” may be formed so as to be in an addressable storage medium, or may be formed so as to operate one or more processors. Thus, for example, the term “unit” may include elements (e.g., software elements, object-oriented software elements, class elements, and task elements), processes, functions, attributes, procedures, subroutines, segments of program code, drivers, firmware, micro-codes, circuits, data, a database, data structures, tables, arrays, or variables. Functions provided by the elements and “units” may be combined into the smaller number of elements and “units”, or may be divided into additional elements and “units”.

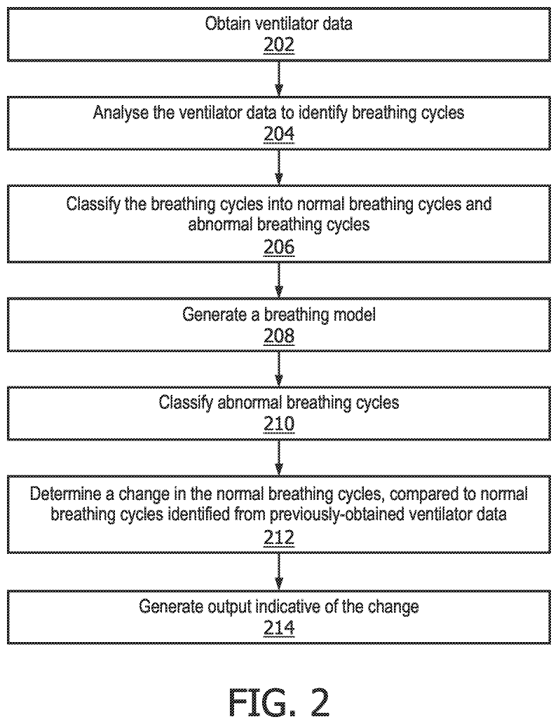

FIG. 2 illustrates a method of tracking breathing cycles of a patient. The method may be performed by or under the control of one or more processors.

At block 202, ventilator data is received from a mechanical ventilator.

At block 204, the ventilator data is analyzed to identify breathing cycles. The analysis may comprise segmenting the ventilator data into breathing cycles. As noted above, the breathing cycles may already be identified in the received ventilator data, thus making the analysis at block 204 optional.

At block 206, the breathing cycles are categorized into normal breathing cycles and abnormal breathing cycles, by applying a machine learning algorithm. Examples are described in more detail with reference to FIG. 3.

At block 208, a patient-specific breathing model is generated. The model may be updated over subsequent periods of time as further ventilator data is obtained. At block 210, the abnormal breathing cycles may be classified into different types of abnormal breathing cycles. For example, the classification may make use of the patient-specific breathing model. However both blocks 208 and 210 are optional.

At block 212, a change in the normal breathing cycle is determined relative to a normal breathing cycle identified in a previous period of time.

At block 214, output indicative of the change is generated.

At least part of the method described with reference to FIG. 2, such as blocks 202, 204, 206, 212, and 214, may be repeated over multiple periods of time.

FIG. 3 illustrates a method of categorizing breathing cycles into normal breathing cycles and abnormal breathing cycles. The method illustrated in FIG. 3 may correspond to the block 206 in FIG. 2, and as such may generally be performed by or under the control of one or more processors.

At block 302, values of a parameter are extracted from received ventilator data. As noted above, the parameter may comprise at least one of a lung parameter and a ventilation parameter. Examples of parameters that may be used in the method include respiratory rate (RR), tidal volume (VT), positive end expiratory pressure (PEEP), compliance, and resistance. For simplicity, reference is made to extracted values below.

At block 304, a machine learning algorithm is applied to the extracted values where each extracted value may be a data point. The machine learning algorithm may be an unsupervised learning method that separates the extracted values into two or more groups, such that the parameter values in the same group have similar properties and extracted values in different groups have different properties in some sense. Examples of machine learning algorithms that may be used include, but are not limited to, K-Means Clustering, Mean-Shift Clustering, Density-Based Spatial Clustering of Applications with Noise (DBSCAN), Gaussian Mixture Models (GMM), and Hierarchical Agglomerative Clustering (HAC).

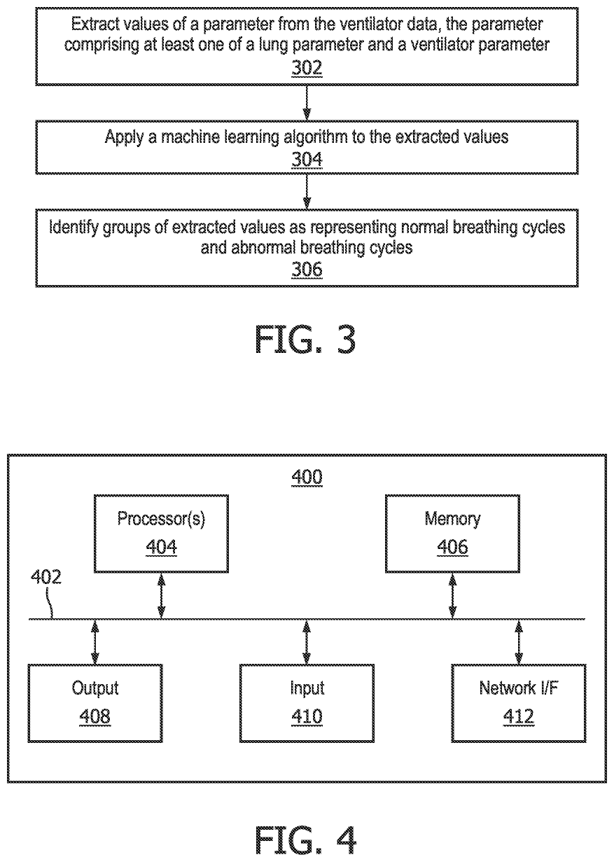

At block 306, the groups of data points (extracted values) obtained at block 304 are identified as representing parameter values of normal breathing cycles and extracted values of abnormal breathing cycles. For example, labels from labelled parameter values may be applied to unlabeled extracted values within the same group. There may be significantly fewer labelled extracted values than unlabeled extracted values. Other ways of differentiating between extracted values of normal breathing cycles and abnormal breathing cycles are also possible. For example, the extracted values of normal breathing cycles may be the most frequently occurring ones, so that the group with the largest number of extracted values may be identified as representing the normal breathing cycles FIG. 4 is a hardware block diagram of a computing device 400, such as the apparatus illustrated in FIG. 1, and which may be used to implement the methods illustrated in FIGS. 2 and 3. The computing device 400 comprises one or more processors 404 and memory 406. Optionally, the computing device 400 may also include a network interface 412. The network interface 412 may be connected to a network, such as the Internet, and is connectable to other such computing devices via the network. The network interface 412 may control data input/output from/to other apparatus via the network. For example, the network interface 412 may provide a connection to the mechanical ventilator 101 or the databases 110, 114, 120 illustrated in FIG. 1. Such a connection may be a wired connection, a wireless connection, or combination of wired and wireless connection. Where the computing device 400 is part of a mechanical ventilator, the network interface 412 may be substituted or complemented by an interface to connect to other components of the mechanical ventilator. In some examples, the processor 404 and memory 406 of the computing device 400 may be the same as those of the mechanical ventilator. In such cases the interface may comprise the bus 402 that connects the components to one another.

The processor 404 is configured to control the computing device and execute processing operations, for example executing code stored in the memory to implement the various functions of the units described with reference to FIG. 1 or to perform the methods described with reference to FIGS. 2 and 3. As referred to herein, a processor may include one or more general-purpose processing devices such as a microprocessor, central processing unit, or the like. The processor may include a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor may also include one or more special-purpose processing devices such as an application specific integrated circuit (ASIC), a field programmable gate array (FPGA), a digital signal processor (DSP), network processor, or the like. In one or more examples, a processor is configured to execute instructions for performing the operations and steps discussed herein.

The memory 406 stores data being read and written by the processor 404. For example, the memory 406 may store received ventilator data, extracted values of parameters (e.g., values of lung parameters and/or values of ventilation parameters), a machine learning algorithm that is applied to the extracted values, output generated by the machine learning algorithm, a patient specific breathing model, etc. The memory 406 may include a computer readable medium, which term may refer to a single medium or multiple media (e.g., a centralized or distributed database and/or associated caches and servers) configured to carry computer-executable instructions or have data structures stored thereon. Computer-executable instructions may include, for example, instructions and data accessible by and causing a general-purpose computer, special purpose computer, or special purpose processing device (e.g., one or more processors) to perform one or more functions or operations. Thus, the term “computer-readable storage medium” may also include any medium that is capable of storing, encoding or carrying a set of instructions for execution by the machine and that cause the machine to perform any one or more of the methods of the present disclosure. The term “computer-readable storage medium” may accordingly be taken to include, but not be limited to, solid-state memories, optical media and magnetic media. By way of example, and not limitation, such computer-readable media may include non-transitory computer-readable storage media, including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices).

Optionally, the computing device 400 also includes one or more input devices 410 such as a keyboard and mouse. These may enable a user to input data and instructions to the computing device. The computing device 400 may also include one or more output devices 408 such as a visual output device (e.g., a display such as one or more monitors, a light source such as an LED or bulb), an audio output device (e.g., a speaker), or any other form of output device. For example, the breathing cycles may be displayed as waveforms on a display. Visual alerts may also be displayed on a display. Audible alerts may be output using the speaker.

To demonstrate the principles of the techniques described herein, and in particular the possibility of capturing changes in breathing cycles obtained from continual monitoring of a patient without the use of hold manoeuvres, simulated data was analyzed to assess changes in ventilatory waveform characteristics and respiratory mechanics using the Trilogy EVO algorithm described in B. Truschel et al. “Trilogy Evo dynamic lung parameters,” white paper Koninklijke Philips N.V., 2019, the entire contents of which are incorporated herein by reference.

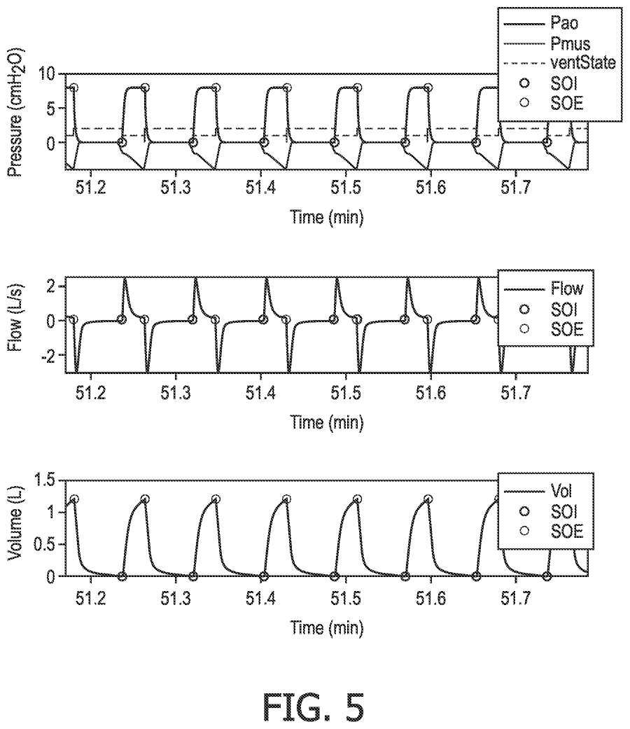

The simulated data is exemplified in the top and middle charts of FIG. 5. The simulated data was obtained using a cardio-respiratory model based on F. Hsing-Hua and M. Khoo, “PNEUMA—a comprehensive cardiorespiratory model,” Proceedings of the Second Joint EMBS/BMES Conference, Houston, Tex., USA, Oct. 23-26, 2002, the entire contents of which are incorporated herein by reference, and Albanese, Antonio et al. “An integrated mathematical model of the human cardiopulmonary system: model development.” American Journal of Physiology-Heart and Circulatory Physiology, Vol. 310, number 7, 2016, the entire contents of which are incorporated herein by reference. It will be understood that these models are used merely for illustrative purposes, and that other models may be utilized instead.

The top and middle graphs of FIG. 5 show representative simulated pressure and flow data with breath segmentation, where “Pao” is airway pressure, “Pmus” is patient respiratory muscle effort, “SOT” is start of inhalation, and “SOE” is start of exhalation. The simulation in FIG. 5 focuses on physiological changes resembling expiratory flow limitation (EFL) in chronic obstructive pulmonary disease (COPD) patients. The physiological feedback loop given by the autonomic control of the cardiorespiratory system, chemoreflex, and state-related control of respiration is considered in the simulations. The traits of respiratory mechanics follow the ideas in P. Barbini et al., “Nonlinear Mechanisms Determining Expiratory Flow Limitation in Mechanical Ventilation: A Model-Based Interpretation,” Annals of Biomedical Engineering 31, 908-916 (2003), the entire contents of which are incorporated herein by reference, ranging from normal to severe EFL.

Using the simulated pressure and flow waveforms shown in top and middle graphs of FIG. 5, the following analysis was carried out:

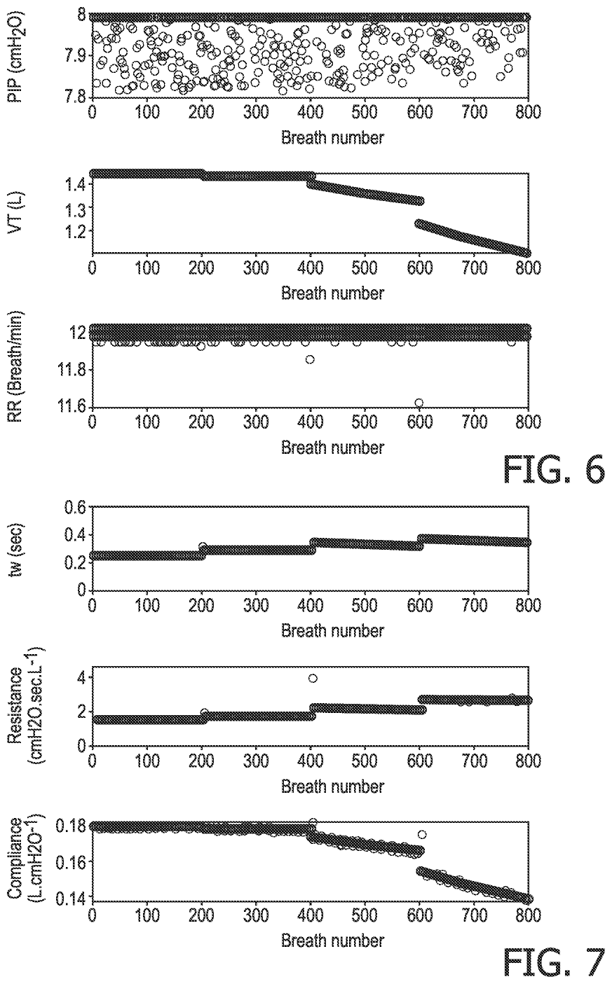

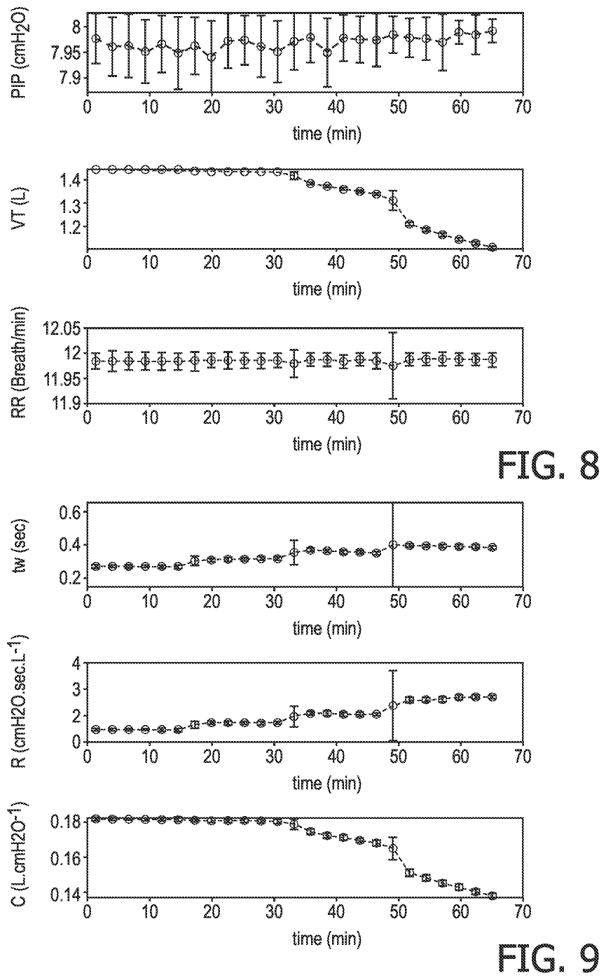

(1) The start and end of inspiration were determined from the ventilatory flag data. As described above, ventilatory flag data may be provided by mechanical ventilators. However, such data may be obtained by waveform analysis instead.
(2) Breath-by-breath integration was carried out to calculate the volume waveform. The results are shown in the bottom graph of FIG. 5.
(3) The ventilation parameters of tidal volume (VT), respiratory rate (RR), and peak inspiratory pressure (PIP) of every breath were calculated. Thresholding based on breath properties is applied as follows: PIP_min=6; PIP_max=10; VT_min=0.5; VT_max=1.5; RR_min=10; RR_max=20. The results are shown in the respective graphs FIG. 6.
(4) The Trilogy EVO algorithm was used to calculate the respiratory lung parameters of respiratory resistance (R), compliance (C), and time constant (tw). The results are shown in the respective graphs of FIG. 7.
(5) To capture general trends across time, the calculated ventilation parameters and respiratory lung parameters shown in FIGS. 6 and 7 were binned (25 bins). The results are shown in FIGS. 8 and 9 respectively.

With reference to FIGS. 8 and 9, since the ventilation mode was pressure support and the patient respiratory drive was a fixed waveform both the peak inspiratory pressure (PIP) and respiratory rate (RR) are constant during the simulation time, as can be seen from the top and bottom graphs of FIG. 8. However, the middle graph of FIG. 8 shows that there is a drop in the tidal volume (VT), which is attributed to the observed increase in resistance (R) corresponding to the four different expiratory flow limitation (EFL) stages that can be seen in the middle graph of FIG. 9. While at the end of simulation the resistance (R) increased by 100%, the compliance (C) only decreased by 22%, leading to an overall increase of 41% in the respiratory time constant (tw).

Thus, the simulation shows that it may be possible to capture trends from waveform analysis of data representing continual (and in this case also non-invasive) mechanical ventilator support of a patient over time. In the simulation describe above, a single set of 800 breathing cycles is used for convenience. However, it will be understood that a corresponding data set obtained from actual ventilator support of a patient may be divided into subsets. For example, such subsets may comprise ventilator data corresponding to 100 breathing cycles or 10-minute periods. In other words, the simulated data corresponding to the time period between 50 and 60 minutes in FIGS. 8 and 9 may be considered as a period of time, and the time period between 10 and 20 minutes in FIGS. 8 and 9 may be considered as a previous period of time. A normal breathing cycle may be identified in each period of time, and the change in breathing cycle may be detected, using the techniques described above. It will be appreciated that these are merely examples and that other subdivisions are also possible.

What has been described and illustrated herein is an example along with some of its variations. The terms, descriptions and figures used herein are set forth by way of illustration only and are not meant as limitations. Many variations are possible within the spirit and scope of the subject matter, which is intended to be defined by the following claims, and their equivalents, in which all terms are meant in their broadest reasonable sense unless otherwise indicated.

1. An apparatus, comprising a processor and memory storing instructions that, when executed by the processor, cause the processor to:
receive ventilator data obtained from continual mechanical ventilatory support of a patient over a period of time,
analyze the ventilator data to identify a plurality of breathing cycles,
classify the plurality of breathing cycles into normal breathing cycles and abnormal breathing cycles, using a machine learning algorithm,
detect a change in the normal breathing cycles, compared to normal breathing cycles identified by the apparatus based on ventilator data obtained from continual mechanical ventilatory support of the patient over a previous period of time, and
generate output indicative of the change in the normal breathing cycles.

2. The apparatus according to claim 1, wherein the instructions further cause the processor to:
generate a respiratory model specific to the patient, based at least on the received ventilator data corresponding to the normal breathing cycles.

3. The apparatus according to claim 1, wherein the instructions further cause the processor to:
analyze the abnormal breathing cycles, using at least one of:
the normal breathing cycles of the patient over the period of time as a reference,
predetermined data describing breathing cycles, and
a predetermined respiratory model describing patient breathing cycles.

4. The apparatus according to claim 1, wherein to classify the plurality of breathing cycles into normal and abnormal breathing cycles the instructions cause the processor to:
extract values of a parameter from the received ventilator data, the parameter comprising at least one of a lung parameter and a ventilation parameter,
apply the machine learning algorithm to the extracted values, to obtain a plurality of groups of extracted values, and
designate at least one group of extracted values as representing the normal breathing cycles, and at least one group of extracted values as representing the abnormal breathing cycles.

5. The apparatus according to claim 4, wherein the designation is based on at least one of:
a numeric property of each of the groups,
a manually generated annotation of the ventilator data, and
predetermined data describing a relationship between values of the parameter and types of breathing cycle.

6. The apparatus according to claim 1, wherein to identify the plurality of breathing cycles from the received ventilator data the instructions cause the processor to:
segment the received ventilator data into a plurality of data segments, each data segment corresponding to an individual breathing cycle.

7. The apparatus according to claim 1, wherein the instructions further cause the processor to:
identify a fraction of the breathing cycles in the ventilator data that are normal breathing cycles, the output data including an indication of the fraction.

8. The apparatus according to claim 1, wherein the ventilator data is received in real time or near real time.

9. The apparatus according to claim 1, wherein the instructions further cause the processor to:
determine a change in the respiratory status of the patient based on the change in the normal breathing cycles, and wherein the output is indicative of the change in the respiratory status.

10. The apparatus according to claim 1, wherein the output comprises an alarm signal indicating that the change in the normal breathing cycles satisfies an alarm condition.

11. A mechanical ventilator including the apparatus according to claim 1.

12. A computer-implemented method, comprising:
receiving ventilator data obtained from continual mechanical ventilatory support of a patient over a period of time;
analyzing the ventilator data to identify a plurality of breathing cycles;
classifying the plurality of breathing cycles into normal breathing cycles and abnormal breathing cycles, using a machine learning algorithm;
detecting a change in the normal breathing cycles, compared to normal breathing cycles identified based on ventilator data obtained from continual mechanical ventilatory support of the patient over a previous period of time; and
generating output indicative of the change in the normal breathing cycles.

13. A computer readable medium storing instructions that, when executed by a processor, cause the processor to:
receive ventilator data obtained from continual mechanical ventilatory support of a patient over a period of time,
analyze the ventilator data to identify a plurality of breathing cycles,
classify the plurality of breathing cycles into normal breathing cycles and abnormal breathing cycles using a machine learning algorithm,
detect a change in the normal breathing cycles, compared to normal breathing cycles identified based on ventilator data obtained from continual mechanical ventilatory support of the patient over a previous period of time, and
generate output indicative of the change in the normal breathing cycles.
